(19) United States
(12) Patent Application Publication
(10) Pub. No.: US 2026/0207918 A1
(43) Pub. Date: Jul. 23, 2026

(54) Systems for Authentication, Identification, and Detection of Disinfection in Medical Devices

(71) Applicant: Becton, Dickinson and Company, Franklin Lakes, NJ (US)

(72) Inventors: Zachary Jarrod Traina, Denville, NJ (US); Jack Balji, Mahwah, NJ (US); Austin Jason McKinnon, Herriman, UT (US); Akiva Wolf Kirschner, Chicago, IL (US); Serge Tsalyuk, New Hope, PA (US); Mitchell L. Thompson, Exton, PA (US)

(21) Appl. No.: 19/141,131

(22) PCT Filed: Jan. 2, 2024
(86) PCT No.: PCT/US2024/010022

§ 371 Date: Jun. 19, 2025

Related U.S. Application Data
(60) Provisional application No. 63/478,215, filed on Jan. 3, 2023.
(60) Provisional application No. 63/479,089, filed on Jan. 9, 2023.

(51) Int. Cl. A61M 39/16; A61M 39/10

(52) U.S. Cl. CPC A61M 39/16; A61M 2039/1005; A61M 2205/332

(57) ABSTRACT

A system may include an optical reader device associated with a medical device, such that when the optical reader device is in one of a plurality of predetermined states, the optical reader device provides one of a plurality of signals associated with the plurality of predetermined states, providing an indication that the medical device is known and further providing an indication of the type of known medical device.

### CROSS-REFERENCE TO RELATED APPLICATIONS

The present application claims priority to U.S. Provisional Application No. 63/478,215 entitled “System for In-Situ, Real Time Optical Authentication and Identification of Infusion Disposables” filed Jan. 3, 2023, the present application also claims priority to U.S. Provisional Application No. 63/479,089 entitled “Piezoelectric Film Sensor for Detecting Disinfection of Connectors in a Medical Administration System” filed Jan. 9, 2023, the entire disclosures of both of which are incorporated by reference herein.

### BACKGROUND

Vascular access management involves many nursing tasks and decisions throughout a patient's stay in care including selecting correct catheters, gathering supplies and preparing catheter insertion sites, inserting catheters, maintaining inserted catheters (e.g., checking insertion sites, changing dressings, changing tubing and connectors, flushing, delivering medications, etc.), and removing catheters. Failures and challenges associated with vascular access management include catheter-related bloodstream infections (CRBSIs), occlusions, unnecessary vascular device usage, blood vessel damage, catheter dislodgement, phlebitis, medication errors, lack of first stick or insertion success, infiltration/extravasation, treatment delays, and/or the like, which may lead to premature removal of catheters, increased healthcare costs, and lower patient satisfaction.

In attempting to improve compliance to all aspects of catheter care, it is desirable to provide the ability to detect or identify the types of devices provided and connected to each other in a catheter assembly, with such detection preferably being done in an automated manner. For example, it is beneficial for a clinician to know the types of devices connected to a needleless access connector (NAC) that provides a fluid connection to an indwelling catheter, as such knowledge conveys clinical benefits such as whether the NAC is capped or uncapped, whether the NAC has or has not been flushed and/or when the NAC is attached to an infusion pump, as non-limiting examples.

It is recognized that some devices or components, such as devices used with a catheter assembly for example, may be provided as disposable or single-use medical devices that are intended to be used once and then be disposed. With such single-use medical devices, it is desirable to not only be able to identify the type of the device, but it is also desirable to be able to determine whether the device is un-used or has been previously used. Visually observing a medical device may not provide accurate information with regard to whether the medical device is used or un-used, and the medical device will typically lack an indication as to whether the medical device has been used before or if the medical device has been reprocessed after a procedure. For instance, a user, such as a doctor, may visually inspect a label on the packaging of a medical device at the time that the packaging is removed from the medical device. In such an instance, the doctor may use the medical device right away after removing the packaging or, conversely, may not administer a medication from the medical device at the time that the packaging is removed. When another user attempts to use the medical device, the other user may not be aware of the status of the medical device with regard to use, and the visual observation of the medical device by itself may not be accurate with regard to whether the medical device was used. Further, the medical device may go unused after the packaging is removed and may be wasted if the status of the medical device with regard to use is not clear.

Therefore, it is desirable to provide a system and method that provides for the automated determination or detection of the types of devices used and/or connected together in, for example, a catheter assembly, including the type of a disposable connected to a NAC. It also desirable for such a system and method to detect whether a disposable is in an un-used condition or a used condition, to prevent waste and/or to prevent the reuse of a contaminated disposable.

In addition, catheter-related bloodstream infections are caused by bacteria/fungi in patients with intravascular catheters. These infections are a significant cause of illness and excess medical costs. Various guidelines and regulations prescribe practices to limit catheter-related bloodstream infections in hospital, outpatient, and home care settings. These guidelines and regulations address issues such clinician hand hygiene, catheter site care, admixture preparation, and connector cleanliness. Despite these guidelines, catheter-related bloodstream infections continue to remain prevalent.

Scrubbing of connectors is an important component of infection prevention, but proper scrubbing can be difficult to quantify and verify. As scrubbing is performed manually by the clinician, the resulting cleanliness of connectors is susceptible to inconsistency, and, in the worst case, inadequacy that places the patient at risk for infection. Therefore, a need exists for connectors which include features for validating that sufficient scrubbing has been performed to mitigate the risk of blood infection from connector contamination.

### SUMMARY

Accordingly, provided are improved systems, devices, products, apparatus, and/or methods for determining a characteristic of a medical device based on an optical reader device.

In accordance with an embodiment of the present invention, a medical device management system includes at least one processor programmed or configured to receive motion data associated with motion of a medical device, determine whether the data associated with motion of the medical device satisfies a threshold, receive optical data associated with an optical reading of a target based on determining that the data associated with motion of the medical device satisfies the threshold, determine a state of the medical device based on the data associated with the reading of the target, and perform an action based on the state of the medical device.

In accordance with an embodiment of the present invention, the system further includes an emitting device, wherein the at least one processor is further programmed or configured to activate the emitting device based on determining that the data associated with motion of the medical device satisfies the threshold. When receiving optical data associated with the optical reading of the target, the at least one processor is programmed or configured to receive optical data associated with an optical reading of a target based on activating the emitting device.

In accordance with an embodiment of the present invention, the system further includes an optical reader device, wherein the data associated with a reading of the target includes data associated with reading of the optical reader device. The at least one processor is further programmed or configured to determine whether the data associated with the reading of the optical reader device corresponds to a predetermined state of a plurality of predetermined states, and when performing the action, the at least one processor is programmed or configured to perform the action based on based on determining that the data associated with the reading of the optical reader device corresponds to a predetermined state of the plurality of predetermined states.

In accordance with an embodiment of the present invention, the system further includes a motion sensing device, wherein the motion sensing device includes a piezoelectric sensor and wherein the piezoelectric sensor is attached to the medical device. When receiving the data associated with motion of the medical device, the at least one processor is programmed or configured to receive data associated with motion of the medical device from the motion sensing device.

In accordance with an embodiment of the present invention, the at least one processor is further programmed or configured to forego activating the emitting device based on determining that the data associated with motion of the medical device does not satisfy the threshold.

In accordance with an embodiment of the present invention, the emitting device comprises a light-emitting diode (LED), and the optical reader device is a photodiode.

In accordance with an embodiment of the present invention, when determining whether the optical data associated with the optical reading of the target corresponds to the predetermined state of the plurality of predetermined states, the at least one processor is programmed or configured to compare the optical data associated with the optical reading of the target to at least one predetermined state of the plurality of predetermined states, and determine that the optical data associated with the optical reading of the target corresponds to the predetermined state of the plurality of predetermined states based on comparing the data associated with the optical reader device to the at least one predetermined state of the plurality of predetermined states.

In accordance with an embodiment of the present invention, the at least one processor is further programmed or configured to determine whether the optical data associated with the optical reading of the target corresponds to a predetermined state of a plurality of predetermined state, wherein each predetermined state of the plurality of predetermined states comprises a characteristic of the medical device, wherein the characteristic of the medical device includes an indication of a type of the medical device. When determining a state of the medical device, the at least one processor is programmed or configured to determine a type of the medical device, and determine an indication of whether the medical device has been used.

In accordance with an embodiment of the present invention, when performing the action, the at least one processor is programmed or configured to provide an indication of the state of the medical device based on the type of the medical device and the indication of whether the medical device has been used.

In accordance with an embodiment of the present invention, when determining the state of the medical device, the at least one processor is programmed or configured to compare the data associated with motion of the medical device to a threshold associated with use of a medical device, and determine that the data associated with motion of the medical device satisfies the threshold. When performing the action, the at least one processor is programmed or configured to provide an indication that the medical device has been used based on determining that the data associated with motion of the medical device satisfies the threshold.

In accordance with an embodiment of the present invention, when performing the action, the at least one processor is programmed or configured to receive acoustic data from the data associated with motion of the medical device, and determine whether the medical device has been used based on a signature of the acoustic data.

In accordance with an embodiment of the present invention, the signature of the acoustic data comprises one of an amplitude and a pattern of the acoustic data.

In accordance with an embodiment of the present invention, a system includes an emitting device, an optical reader device, a motion sensing device, and a medical device management system including at least one processor programmed or configured to receive data associated with motion of the medical device from the motion sensing device, determine whether the data associated with motion of the medical device satisfies a threshold, activate the emitting device based on determining that the data associated with motion of the medical device satisfies the threshold, receive data associated with an optical reading of a target by the optical reader device based on activating the emitting device, determine a state of the medical device based on the data associated with the optical reading of the target, and perform an action based on the state of the medical device.

In accordance with an embodiment of the present invention, the motion sensing device includes a piezoelectric sensor and wherein the piezoelectric sensor is attached to the medical device.

In accordance with an embodiment of the present invention, the at least one processor is further programmed or configured to forego activating the emitting device based on determining that the data associated with motion of the medical device does not satisfy the threshold.

In accordance with an embodiment of the present invention, the emitting device comprises a light-emitting diode (LED), and the optical reader device is a photodiode.

In accordance with an embodiment of the present invention, when determining the state of the medical device, the at least one processor is further programmed or configured to determine whether the data associated with the optical reading of the target corresponds to a predetermined state of a plurality of predetermined states.

In accordance with an embodiment of the present invention, when determining a state of the medical device, the at least one processor is programmed or configured to determine a type of the medical device, or determine an indication of whether the medical device has been used.

In accordance with an embodiment of the present invention, when performing the action, the at least one processor is programmed or configured to provide an indication of the state of the medical device based on the type of the medical device and the indication of whether the medical device has been used, the indication comprising a tangible signal indicating the state of the medical device.

In accordance with an embodiment of the present invention, a system includes a housing, an emitting device, an optical reader device, a motion sensing device, and a medical device management system. The emitting device, the optical reader device, and the motion sensing device are contained in the housing, and the medical device management system comprises at least one processor programmed or configured to receive data associated with motion of the medical device from the motion sensing device, determine whether the data associated with motion of the medical device satisfies a threshold, activate the emitting device based on determining that the data associated with motion of the medical device satisfies the threshold, receive data associated with an optical reading of a target by the optical reader device based on activating the emitting device, determine a state of the medical device based on the data associated with the optical reading of the target, and perform an action based on the state of the medical device.

In accordance with an embodiment of the present invention, a connector for a catheter assembly includes a body defining longitudinal axis and a lumen, a first fitting at a first end of the body, a second fitting at a second end of the body, a flexible seal disposed in the first fitting for preventing contamination in the first fitting, and a scrubbing sensor disposed on the body a predetermined distance from an end of the first fitting. The scrubbing sensor is configured to generate an electrical signal in response to a strain in the body, wherein the strain corresponds to at least one of a force applied to the first fitting and a torque applied to the first fitting.

In accordance with an embodiment of the present invention, the scrubbing sensor includes a piezoelectric film.

In accordance with an embodiment of the present invention, the piezoelectric film is made of polyvinylidene fluoride.

In accordance with an embodiment of the present invention, the scrubbing sensor is arranged on the body such that a primary axis of the piezoelectric film is at an angle relative to the longitudinal axis.

In accordance with an embodiment of the present invention, the angle is approximately 30°.

In accordance with an embodiment of the present invention, the angle is approximately 45°.

In accordance with an embodiment of the present invention, the angle is in a range of approximately 10° to approximately 80°.

In accordance with an embodiment of the present invention, the angle is in a range of approximately 20° to approximately 70°.

In accordance with an embodiment of the present invention, the angle is in a range of approximately 30° to approximately 60°.

In accordance with an embodiment of the present invention, the angle is in a range of approximately 40 ° to approximately 50°.

In accordance with an embodiment of the present invention, the scrubbing sensor is arranged on the body such that the primary axis of the piezoelectric film is parallel to the longitudinal axis.

In accordance with an embodiment of the present invention, the connector further includes an electrical module having one or contacts configured to engage one or more contact surfaces of the scrubbing sensor to receive the voltage signal from the scrubbing sensor.

In accordance with an embodiment of the present invention, the scrubbing sensor includes at least two sectors spaced apart from one another about a circumference of the body.

In accordance with an embodiment of the present invention, each of the at least two sectors is configured to output an independent voltage signal.

In accordance with an embodiment of the present invention, the at least two sectors are spaced 90° apart from one another about the circumference of the body.

In accordance with an embodiment of the present invention, the connector further includes a cover protecting the scrubbing sensor and the electrical module.

In accordance with an embodiment of the present invention, the first fitting includes a female luer fitting.

In accordance with an embodiment of the present invention, the second fitting includes a male luer fitting.

Further details and advantages of the various examples described in detail herein will become clear upon reviewing the following detailed description of the various examples in conjunction with the accompanying drawing figures.

### BRIEF DESCRIPTION OF THE DRAWINGS

Additional advantages and details are explained in greater detail below with reference to the exemplary embodiments that are illustrated in the accompanying schematic figures, in which:

FIG. 1 is a diagram of a non-limiting embodiment or aspect of an environment in which systems, devices, products, apparatus, and/or methods, described herein, may be implemented according to the principles of the present disclosure;

FIG. 2 is a diagram of a non-limiting aspect or embodiment of components of one or more devices and/or one or more systems of FIG. 1;

FIG. 3 is a flowchart of a non-limiting aspect or embodiment of a process for determining a state of a medical device;

FIG. 4 is a diagram of graph 400 representing data associated with motion sensing device according to non-limiting embodiments.

FIGS. 5A-5D are diagrams of a non-limiting embodiment of a system for medical device authentication and identification;

FIG. 6 is a diagram of an implementation of a non-limiting embodiment of a system for medical device authentication and identification, including an optical reader device, in proximity to a syringe;

FIG. 7 is a diagram of an implementation of a non-limiting embodiment of a system for medical device authentication and identification attached to another system for medical device authentication and identification;

FIG. 8 is a diagram of an implementation of a non-limiting embodiment of a system for medical device authentication and identification, including an optical reader device and an emitting device, in contact with a cap device; and

FIG. 9 is a graph representing different time-decay profiles of different taggants as disclosed herein.

FIG. 10 is a perspective view of a connector according to an embodiment of the present disclosure;

FIG. 11 is a perspective view of the connector of FIG. 10, with a cover and dust cap thereof removed;

FIG. 12 is a perspective view of the connector of FIG. 10, with an electronics module thereof removed;

FIG. 13 is a front perspective view of a connector according to an embodiment of the present disclosure;

FIG. 14 is front perspective view of the connector of FIG. 13;

FIG. 15 is a side view of the connector of FIG. 13;

FIG. 16 is a top view of the connector of FIG. 13;

FIG. 17 is a top view of a scrubbing sensor of the connector of FIGS. 10-16, according to an embodiment of the present disclosure;

FIG. 18 is a graph showing the variance in stress around the cylinder due to a single force applied in a single direction.

FIG. 19 is a graph showing sensitivity of the scrubbing sensor as a function of the angular orientation of the piezoelectric film of the scrubbing sensor;

FIG. 20 is a graph showing voltage output of the scrubbing sensor as a function of the angle at which a force is applied to the connector;

FIG. 21 is a graph showing voltage output of two independent sectors of the scrubbing sensor, arranged 90° apart about the connector, as a function of the angle at which a force is applied to the connector; and

FIG. 22 is a graph showing the impact of the lengthening of the piezo-electric sensor.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments. Hence, specific dimensions and other physical characteristics related to the embodiments disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

All numbers used in the specification and claims are to be understood as being modified in all instances by the term “about”. The terms “approximately”, “about”, and “substantially” mean a range of plus or minus ten percent of the stated value. Further, the term “substantially equal” and like terms mean that the compared values or dimensions are within a range of plus or minus ten percent of one another.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” As used in the specification and the claims, the singular form of “a,” “an,” and “the” include plural referents, such as unless the context clearly dictates otherwise. Additionally, Furthermore, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

The terms “first”, “second”, and the like are not intended to refer to any particular order or chronology, but refer to different conditions, properties, or elements.

As used herein with reference to an injection apparatus such as a syringe, the term “proximal” refers to an end of the apparatus farthest from the outlet, or to a direction toward the end of the apparatus farthest from the outlet. As used herein with reference to an injection apparatus such as a syringe, the term “distal” refers to an end of the device or apparatus closest to the outlet, or to a direction toward the end of the apparatus closest to the outlet.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments, a message may refer to a network packet (e.g., a data packet and/or the like) that includes data.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, equal to the threshold, etc.

Provided are improved devices, systems, methods, and products for accurately determining a state of a medical device based on data associated with a reading of a target, which may refer to the medical device, as read by an optical reader device. Embodiments of the present disclosure may include a medical device management system that includes an optical reader device associated with a motion sensing device and an emitting device. In some non-limiting embodiments, the medical device management system further comprises at least one processor programmed or configured to receive data associated with the motion sensing device, determine whether the data associated with the motion sensing device satisfies a threshold, and perform an action based on determining whether the data associating with the motion sensing device satisfies a threshold. In some non-limiting embodiments, the medical device management system is programmed or configured to receive data associated with optical reader device, determine a characteristic of the medical device based on the data associated with the optical reader device, and perform an action based on the characteristic of the medical device.

In this way, embodiments of the present disclosure provide a system that is able to provide accurate information with regard to a state of the medical device, such as whether the medical device is attached, whether the type of medical device is known or unknown, and if known, a type of the medical device, and with regard to whether the medical device has been used or is unused.

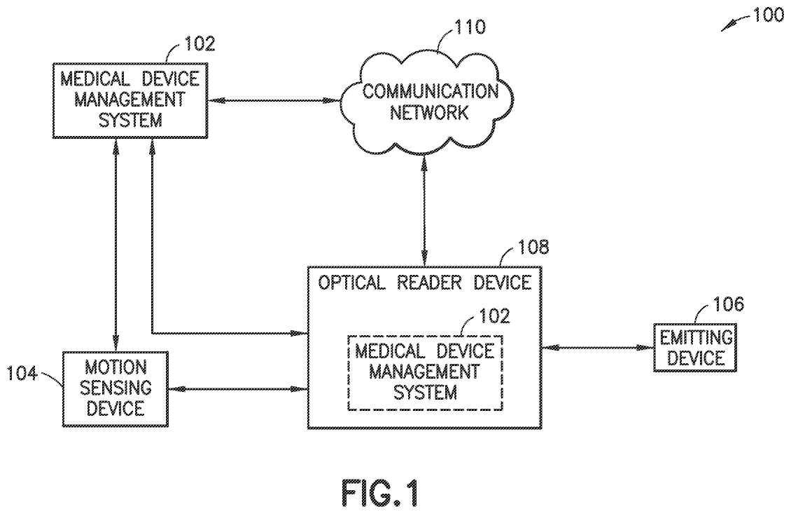

Referring now to FIG. 1, FIG. 1 is a diagram of an example environment 100 in which devices, systems, methods, and/or products described herein may be implemented. As shown in FIG. 1, environment 100 includes medical device management system 102, motion sensing device 104, emitting device 106, optical reader device 108, and communication network 110. Medical device management system 102 may interconnect (e.g., establish a connection to communicate, and/or the like) with motion sensing device 104 via wired connections, wireless connections, or a combination of wired and wireless connections. Medical device management system 102 may interconnect with emitting device 106 and/or optical reader device 108 via wired connections, wireless connections, or a combination of wired and wireless connections. In some non-limiting embodiments, medical device management system 102, motion sensing device 104, emitting device 106, and optical reader device 108 may be combined into a single system or device. For example, medical device management system 102, motion sensing device 104, emitting device 106, and optical reader device 108 may be contained within a housing of a portable (e.g., hand-held) system or device.

Medical device management system 102 may include one or more devices configured to be in communication with optical reader device 108 and motion sensing device 104 via communication network 110. For example, medical device management system 102 may include a server (e.g., a cloud server), a group of servers, a computing device, such as a mobile device (e.g., a smartphone) a tablet computer, a laptop computer, a desktop computer, and/or the like. In some non-limiting embodiments, medical device management system 102 may be configured to be in communication with motion sensing device 104 and optical reader device 108 via direct communication connections (e.g., a communication connection that is independent of a communication network, such as communication network 110), such as a short-range wireless communication connection (e.g., a near-field communication (NFC) communication connection, a radio frequency identification (RFID) communication connection, a Bluetooth® communication connection, an infrared communication connection, etc.) or a wired communication connection (e.g., a connection that uses a cable and universal serial bus (USB) communication protocol). In some non-limiting embodiments, medical device management system 102 may be configured to determine a characteristic of a medical device based on data received from motion sensing device 104. In some non-limiting embodiments, medical device management system 102 may be configured to determine a characteristic of a medical device based on data received from optical reader device 108.

Motion sensing device 104 may include one or more devices configured to generate an output upon disturbance (e.g., mechanical disturbance) of motion sensing device 104 by the application of direct contact with or attachment of a medical device (e.g., a closure device, such as a cap). For example, motion sensing device 104 may include a sensor, such as a piezoelectric sensor device. In some non-limiting embodiments or aspects, motion sensing device 104 may output data associated with disturbance of motion sensing device 104. For example, motion sensing device 104 may output an electrical signal associated with disturbance of motion sensing device 104.

Emitting device 106 may include one or more sources of electromagnetic radiation. In some non-limiting embodiments, emitting device 106 may include a light-emitting diode (LED) configured to emit electromagnetic radiation having a wavelength within the visible light spectrum (e.g. a wavelength in the range of 380 nm to 700 nm). In some non-limiting embodiments, emitting device 106 may be an LED configured to emit electromagnetic radiation having a wavelength within the infrared light spectrum (e.g. a wavelength in the range of 800 nm to 1000 nm). In some non-limiting embodiments, emitting device 106 may be configured to direct electromagnetic radiation in the direction of a medical device (e.g. a closure device, such as a cap) in proximity to or attached to system for medical device authentication and identification. In some non-limiting embodiments or aspects, emitting device 106 may be configured to have at least two states, including a non-activated state in which emitting device 106 is not emitting electromagnetic radiation, and an activated state in which emitting device 106 is emitting electromagnetic radiation.

Optical reader device 108 may include one or more devices configured to be in communication with medical device management system 102 via communication network 110. For example, optical reader device 108 may include a server (e.g., a cloud server), a group of servers, a computing device, such as a mobile device (e.g., a smartphone, a wearable device, etc.), a tablet computer, a laptop computer, a desktop computer, and/or the like. In some non-limiting embodiments, optical reader device 108 may include a photodiode configured to be in connection with medical device management system 102 via a direct communication connection (e.g., a communication connection that is independent of a communication network, such as communication network 110), such as short-range wireless communication connection (e.g., an NFC communication connection, an RFID communication connection, a Bluetooth® communication connection, an infrared communication connection, etc.) or a wired communication connection (e.g., a connection that uses a cable and USB communication protocol). In some non-limiting embodiments or aspects, medical device management system 102 may be a component of or separate from optical reader device 108. In some non-limiting embodiments, optical reader device 108 may be configured to absorb electromagnetic radiation and generate an output received by medical device management system 102.

In some non-limiting embodiments, the medical device may include a device that is used in a medical procedure. For example, the medical device may include a device that is used to establish a connection during a medical procedure, such as a luer device (e.g., a luer connector), a catheter hub, a needleless connector, a port, a manifold, a stopcock, and/or the like. In some non-limiting embodiments, the medical device may include one of a mating medical device (e.g., medical devices that are designed to be connected together) or a set of mating medical devices. For example, the medical device may include a needlefree connector and/or a corresponding cap. Additionally or alternatively, the medical device may include a needlefree connector and/or a syringe (e.g., a flush syringe). In another example, the medical device may include a tubing element and a syringe. In another example, the medical device may include a test cartridge and a reader (e.g., a test cartridge for an ailment, such as a disease or sickness, and a reader). In another example, the medical device may include an oral syringe and oral tubing connector. In another example, the medical device may include a syringe and needle. In some non-limiting embodiments, a medical device (e.g. a medical device associated with motion sensing device 104) may include a container (e.g. medication container), a syringe, an IV assembly, a luer connector, a catheter, a tubing, medication packaging, a closure device (e.g. a cap), and/or the like.

Communication network 110 may include one or more wired and/or wireless networks. For example, communication network 110 may include a cellular network (e.g., a long-term evolution (LTE) network, a third generation (3G) network, a fourth generation (4G) network, a fifth generation (5G), network a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of some or all of these or other types of networks.

The number and arrangement of systems and/or devices shown in FIG. 1 are provided as an example. There may be additional systems and/or devices, fewer systems and/or devices, different systems and/or devices, or differently arranged systems and/or devices than those shown in FIG. 1. Furthermore, two or more systems and/or devices shown in FIG. 1 may be implemented within a single system or a single device, or a single system or a single device shown in FIG. 1 may be implemented as multiple, distributed systems or devices. Additionally or alternatively, a set of systems or a set of devices (e.g., one or more systems, one or more devices) of environment 100 may perform one or more functions described as being performed by another set of systems or another set of devices of environment 100.

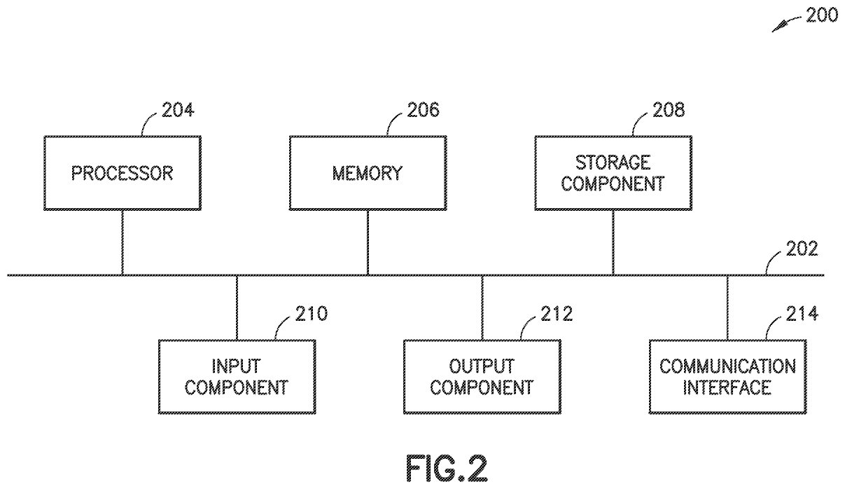

Referring now to FIG. 2, FIG. 2 is a diagram of example components of device 200. Device 200 may correspond to medical device management system 102 (one or more devices of medical device management system 102) and/or optical reader device 108. In some non-limiting embodiments, medical device management system 102 and/or optical reader device 108 may include at least one device 200 or at least one component of device 200. As shown in FIG. 2, device 200 may include a bus 202, a processor 204, a memory 206, a storage component 208, an input component 210, an output component 212, and a communication interface 214.

Bus 202 may include a component that permits communication among the components of device 200. In some non-limiting embodiments, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function.

Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid-state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, etc.). Additionally or alternatively, input component 210 may include a sensor for sensing information (e.g., a global positioning system (GPS) component, an accelerometer, a gyroscope, an actuator, etc.).

Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments described herein are not limited to any specific combination of hardware circuitry and software.

Memory 206 and/or storage component 208 may include data storage or one or more data structures (e.g., a database, and/or the like). Device 200 may be capable of receiving information from, storing information in, communicating information to, or searching information stored in the data storage or one or more data structures in memory 206 and/or storage component 208. For example, the information may include data associated with a real-time mobile device application profile, data associated with a historical mobile device application profile, input data, output data, transaction data, account data, or any combination thereof.

The number and arrangement of components shown in FIG. 2 are provided as an example. In some non-limiting embodiments, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

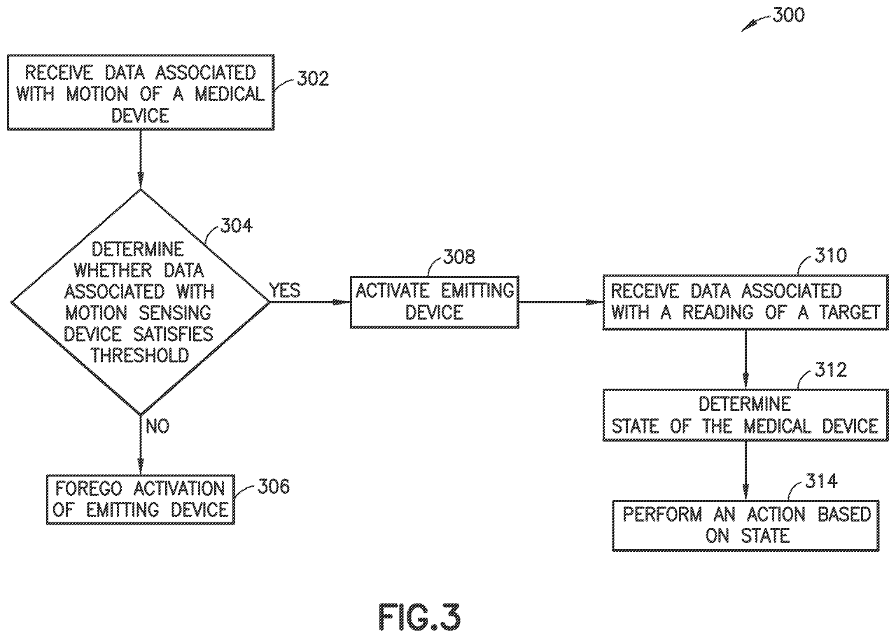

Referring now to FIG. 3, FIG. 3 is a flowchart of a non-limiting embodiment of a process 300 for determining a state (e.g., a characteristic) of a medical device. In some non-limiting embodiments, one or more of the functions described with respect to process 300 may be performed (e.g., completely, partially, etc.) by medical device management system 102. In some non-limiting embodiments, one or more of the steps of process 300 may be performed (e.g., completely, partially, and/or the like) by another device or a group of devices separate from medical device management system 102, such as optical reader device 108, motion sensing device 104, or emitting device 106.

As shown in FIG. 3, at step 302, process 300 may include receiving data associated with a motion sensing device. For example, medical device management system 102 may receive (e.g., receive in real-time) the data associated with motion sensing device 104 from motion sensing device 104. In some non-limiting embodiments, medical device management system 102 may receive motion data associated with motion of a medical device. For example, when receiving the data associated with motion of the medical device, medical device management system 102 may receive data associated with motion of the medical device from the motion sensing device. In some non-limiting embodiments, motion sensing device 104 may be a piezoelectric sensor. The piezoelectric sensor may be associated with (e.g., attached to) the medical device.

In some non-limiting embodiments, motion sensing device 104 may be in contact with (e.g., attached to, positioned on, adhered to, stuck on, applied to a surface of, etc.) the medical device. In some non-limiting embodiments, the data associated with motion sensing device 104 may be an electrical signal generated upon disturbance (i.e. mechanical disturbance) of motion sensing device 104 by the application of contact with or attachment of the medical device. In some non-limiting embodiments, medical device management system 102 may be activated (e.g., processor 204 of system 102 may be changed from an inactive-state to an active-state) when an amount of motion sensed by motion sensing device 104 is above a threshold (e.g., an activation threshold). That is, medical device management system 102 may be activated when a voltage generated by the motion sensing device 104 when (and provided to system 102) is above a threshold for activating system 102, with a threshold voltage corresponding to the threshold level of motion that is sensed by the motion sensing device 104 that causes the voltage to be generated.

As shown in FIG. 3, at step 304, process 300 may include determining whether data associated with motion sensing device 104 satisfies a threshold. In some non-limiting embodiments, medical device management system 102 may determine whether the data associated with motion of the device satisfies a threshold. For example, medical device management system 102 may determine whether signal produced by (e.g., and received by medical device management system 102) motion sensing device 104 satisfies a threshold. In some non-limiting embodiments, medical device management system 102 may receive the signal produced by motion sensing device 104 and compare the signal to a threshold. In one example, medical device management system 102 may determine that the signal produced by motion sensing device 104 satisfies the threshold based on comparing the signal to the threshold. In another example, medical device management system 102 may determine that the signal produced by motion sensing device 104 does not satisfy the threshold based on comparing the signal to the threshold.

For example, medical device management system 102 may determine that data associated with motion sensing device 104 satisfies a threshold, or medical device management system 102 may determine that data associated with motion sensing device does not satisfy a threshold.

As shown in FIG. 3, at step 306 (“No”), process 300 may include foregoing performing an action. For example, medical device management system 102 may forego performing an action. In some non-limiting embodiments, medical device management system 102 may forego performing an action based on determining that the data associated with motion sensing device 104 does not satisfy the threshold. In some non-limiting embodiments, medical device management system 102 may forego activation of emitting device 106. For example, medical device management system 102 may forego activation of emitting device 106 based on determining that the data associated with motion sensing device 104 does not satisfy the threshold. In some non-limiting embodiments, medical device management system 102 may forego receiving data associated with a reading of a target based on determining that the data associated with motion sensing device 104 does not satisfy the threshold.

As shown in FIG. 3, at step 308 (“Yes”), process 300 may include performing an action. For example, medical device management system 102 may perform an action. In some non-limiting embodiments, medical device management system 102 may perform an action based on determining that the data associated with motion sensing device 104 satisfies the threshold. In some non-limiting embodiments, medical device management system 102 may activate emitting device 106. For example, medical device management system 102 may activate emitting device 106 based on determining that the data associated with motion sensing device 104 satisfies the threshold. In some non-limiting embodiments, medical device management system 102 may receive data associated with a reading of a target based on determining that the data associated with motion sensing device 104 satisfies the threshold. In some non-limiting embodiments, medical device management system 102 may perform the action by changing emitting device 106 from an inactivated state (e.g., an off-state) to an activated state (e.g., an on-state).

As shown in FIG. 3, at step 310, process 300 may include receiving data associated with a reading of a target. In some non-limiting embodiments, medical device management system 102 may receive data associated with a reading of the target (e.g., data from a reader device, such as optical reader device 108). In some non-limiting embodiments, the target may include the medical device of which a reading is taken. In some non-limiting embodiments, medical device management system 102 may receive optical data associated with an optical reading of a target (e.g., based on determining that the data associated with motion of the device satisfies the threshold). In some non-limiting embodiments, the optical data associated with an optical reading of the target may include electromagnetic radiation in a visible light spectrum reflected by the target or may include electromagnetic radiation in the infrared spectrum provided by the target In some non-limiting embodiments, when receiving optical data associated with the optical reading of the target, medical device management system 102 may receive optical data associated with an optical reading of a target based on activating emitting device 106. In some non-limiting embodiments, medical device management system 102 may receive the optical data associated with an optical reading of the target from optical reader device 108. In some non-limiting embodiments, data associated with a reading of the target comprises data associated with a reading of the target by optical reader device 108.

In some non-limiting embodiments, optical reader device 108 may be configured to absorb electromagnetic radiation reflected or emitted by a medical device in proximity to or attached to the system for medical device authentication and identification. In some non-limiting embodiments, optical reader device 108 may be configured to provide data associated with the electromagnetic radiation absorbed by optical reader device 108 as an output (e.g., an output signal).

As shown in FIG. 3, at step 312, process 300 may include determining a state of a medical device. For example, medical device management system 102 may determine a state (e.g., a predetermined state) of the medical device. In some non-limiting embodiments, medical device management system 102 may determine a state of the medical device based on the data associated with the reading of the target.

In some non-limiting embodiments, medical device management system 102 may determine a type of the medical device and/or determine an indication of whether the medical device has been used. For example, medical device management system 102 may determine the type of the medical device based on data associated with a reading of a target. Additionally or alternatively, medical device management system 102 may determine an indication of whether the medical device has been used based on data associated with motion of the medical device.

In some non-limiting embodiments, when determining the state of the medical device, medical device management system 102 may compare data associated with motion of the medical device to a condition or signature associated with use of a medical device and may determine that the data associated with motion of the medical device satisfies the condition or signature. In such an example, medical device management system 102 may determine that the state of the medical device is used. In some non-limiting embodiments, when determining the state of the medical device, medical device management system 102 may compare data associated with motion of the medical device to the condition or signature associated with use of a medical device and may determine that the data associated with motion of the medical device does not satisfy the condition or signature. In such an example, medical device management system 102 may determine that the state of the medical device is not used (e.g., new, unused, etc.). According to embodiments, the condition or signature associated with use of a medical device may be a differentiable acoustic signature, with the differentiable acoustic signature having a variation of magnitude, pattern, or type (e.g., one click vs. two clicks for used and un-used devices, respectively). That is, the data associated with motion of the medical device (i.e., a piezo signal) may be analyzed by medical device management system 102 to identify an acoustic signature therein indicative of the use condition of the medical device.

In some non-limiting embodiments, medical device management system 102 may determine whether data associated with a reading of a target (e.g., data associated with optical reader device 108, such as data associated with a reading of a target by optical reader device 108) corresponds to (e.g. matches) a stored value (e.g., of a plurality of stored values) for a type of medical device (e.g., a type of medical device of a plurality of types of medical devices). For example, medical device management system 102 may determine whether the data associated with a reading of a target corresponds to a stored value of a plurality of stored values corresponding to a plurality of known types of medical devices. In some non-limiting embodiments, medical device management system 102 may produce data associated with optical reader device 108 to produce a value that is compared to a plurality of stored values corresponding to a plurality of types (e.g., known types) of medical devices. In such an example, medical device management system 102 may determine that the state of the medical device is type of the medical device.

In some non-limiting embodiments, medical device management system 102 may determine that data associated with a reading of a target matches a stored value corresponding to a type (e.g., a known type) of medical device, and medical device management system 102 may determine the type of medical device (e.g., the type of medical as indicated by the state of the medical device) based on the data associated with the reading of the target. In some non-limiting embodiments, medical device management system 102 may determine that data associated with optical reader device 108 does not match a stored value corresponding to a known type of medical device and medical device management system 102 may not be able to determine that the type of medical device based on the data associated with the reading of the target.

As shown in FIG. 3, at step 314, process 300 may include performing an action based on determining whether data associated with optical reader device 108 matches (i.e. is similar or identical to) a stored value of a plurality of stored values corresponding to a plurality of known medical device types. For example, medical device management system 102 may perform an action based on determining whether data associated with optical reader device 108 matches a stored value of a plurality of stored values corresponding to a plurality of known medical device types. In some non-limiting embodiments, medical device management system 102 may provide an indication that the type of medical device associated with medical device management system 102 is of a known type. In some non-limiting embodiments, medical device management system 102 may transmit the data to a database. In some non-limiting embodiments, medical device management system 102 may transmit the data to an electronic medical record (EMR) system for automatic logging and/or safety confirmation that medication to be administered matches a medication order for an individual.

In some non-limiting embodiments, when performing the action, medical device management system 102 may provide an indication of the state of the medical device based on the type of the medical device and/or the indication of whether the medical device has been used. In some non-limiting embodiments, the indication of the state of the medical device may include a tangible signal in the form of an acoustic signal (e.g., an audible signal), a visible signal, a tactile signal, and/or the like.

In some non-limiting embodiments, when performing the action, medical device management system 102 may provide an indication that the medical device has been used based on determining that the data associated with motion of the medical device satisfies the threshold. Additionally or alternatively, when performing the action, medical device management system 102 may provide an indication of a type of the medical device based on determining that data associated with a reading of a target corresponds to the type of the medical device.

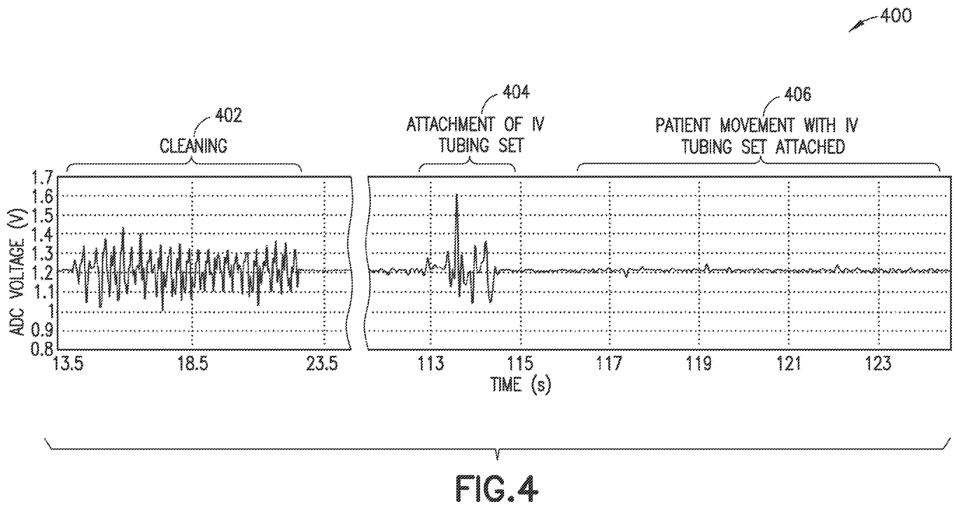

Referring to FIG. 4, FIG. 4 is a diagram of graph 400 representing data associated with motion sensing device according to non-limiting embodiments. As shown in FIG. 4, data associated with motion sensing device 104 may include values, measured in voltage over time, corresponding to disturbance of motion sensing device 104. In some non-limiting embodiments, medical device management system 102 may receive data associated with motion sensing device 104 and determine whether data associated with motion sensing device 104 satisfies a threshold. For example, medical device management system 102 may receive data corresponding to cleaning of medical device management system as shown in section 402 of graph 400, and determine that data corresponding to cleaning 402 as shown in section 402 does not satisfy the threshold. As another example, medical device management system 102 may receive data corresponding to attachment of a medical device as shown in section 404 of graph 400 and determine that data corresponding to attachment of a medical device as shown in 404 satisfies the threshold. As another example, medical device management system 102 may receive data corresponding to movement of a patient's arm as shown in section 406 of graph 400, to which medical device management system 102 is attached, and medical device management system 102 may determine that data corresponding to movement of the patient's arm as shown in section 406 does not satisfy the threshold.

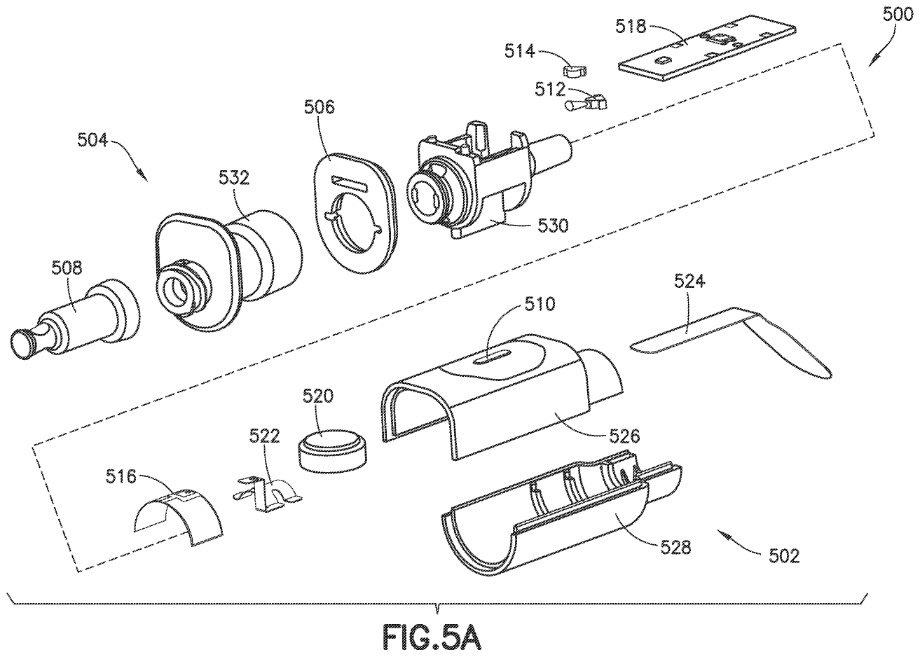

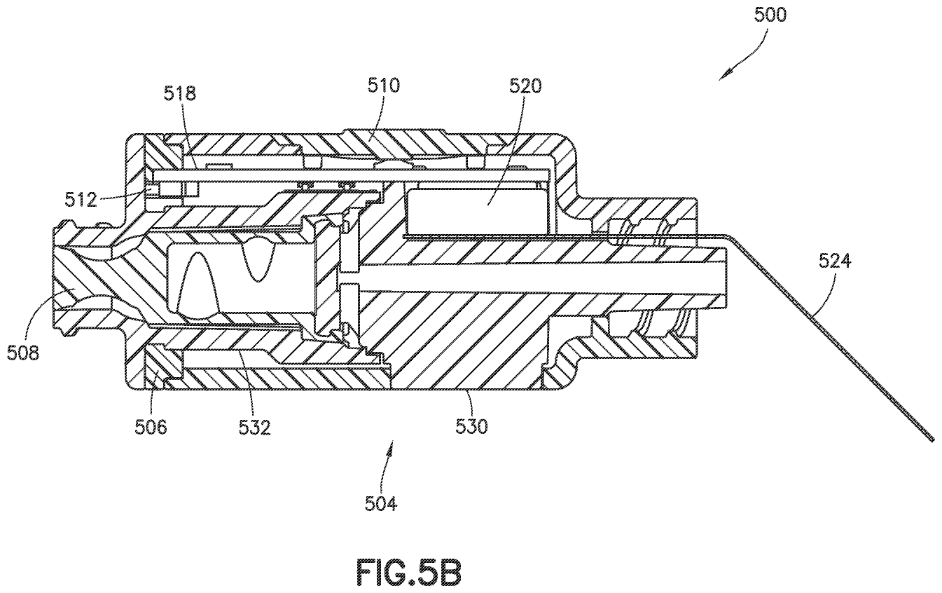

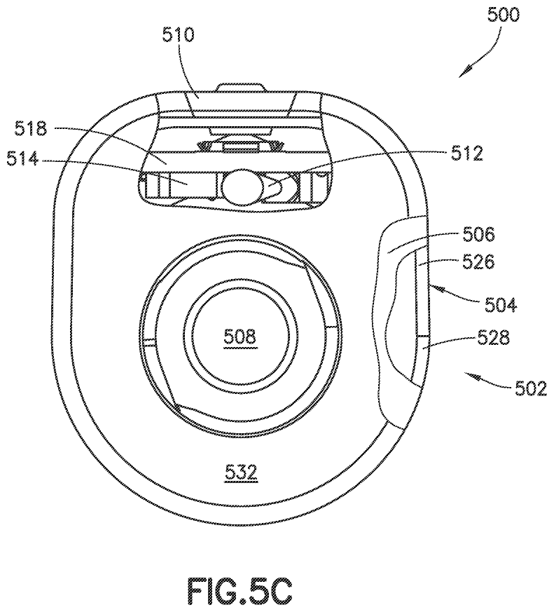

Referring to FIGS. 5A-5D, FIGS. 5A-5D are diagrams of non-limiting embodiments of a system for medical device authentication and identification 500. In some non-limiting embodiments, the system for medical device authentication and identification 500 may be associated with a medical device. For example, the medical device may be a needleless connector, according to an exemplary embodiment, or may be syringe, an IV assembly, a luer connector, a catheter, tubing, a container, medication packaging, a closure (e.g., a cap), and/or the like.

Referring to FIG. 5A, FIG. 5A is a disassembled view of the system for medical device authentication and identification 500. As shown in FIG. 5A, system for medical device authentication and identification 500 may include housing 502, front section 504, aperture plate 506, deformable valve 508 (e.g., split septum valve), tactile switch 510, motion sensing device 516, emitting device 512, optical reader device 514, circuit board 518, battery 520, contact piece 522, and battery insulator 524. In some non-limiting embodiments, emitting device 512, optical reader device 514, and motion sensing device 516 are contained in the housing 502.

In some non-limiting embodiments, motion sensing device 512 may be the same as or similar motion sensing device 104. In some non-limiting embodiments, emitting device 512 may be the same as or similar to emitting device 106. In some non-limiting embodiments, optical reader device 514 may be the same as or similar to optical reader device 108.

In some non-limiting embodiments, housing 502 may include upper housing 526 and lower housing 528. In some non-limiting embodiments, housing 502 may be made of a material that is appropriate based on the use for which housing 502 is designed. For example, the material of housing 502 may include plastic, glass, and/or the like. In some non-limiting embodiments, upper housing 526 and lower housing 528 may be configured to connect together based on a friction fit, an adhesive, an ultrasonic weld, or connectors (e.g., screws). In some non-limiting embodiments, upper housing 526 may be further associated with tactile switch 510.

In some non-limiting embodiments, front section 504 may include inner front section 530 and outer front section 532. In some non-limiting embodiments, front section 504 may be made of a material that is appropriate based on the use for which front section 504 is designed and which can be cleaned. For example, the material of front section 504 may include plastic, glass, and/or the like. In some non-limiting embodiments, outer front section 532 may be configured to receive inner front section 530, such that inner front section 530 is housed inside outer front section 532. In some non-limiting embodiments, outer front section 532 may be configured to receive a medical device. In some non-limiting embodiments, inner front section 530 may be associated with deformable valve 508.

In some non-limiting embodiments, aperture plate 506 may include one or more apertures. In some non-limiting embodiments, aperture plate 506 may be associated with outer front section 532. In some non-limiting embodiments, aperture plate 506 may be configured to direct electromagnetic radiation through apertures which allow electromagnetic radiation to travel from inside medical device management system 102 to outside of medical device management system 102, or vice versa, while blocking internal reflection. In some non-limiting embodiments, aperture plate 506 may be made of a material that is appropriate based on the use for which aperture plate 506 is designed. For example, the material of aperture plate 506 may include plastic, glass, and/or the like.

In some non-limiting embodiments, deformable valve 508 facilitates and controls the passage of fluid from the medical device associated with medical device management system 102 to a patient without interfering with the operation of medical device management system 102. In some non-limiting embodiments, deformable valve 508 may be made of a material that is appropriate based on the use for which deformable valve 508 is designed. For example, the material of deformable valve 508 may include an elastomeric material and/or the like.

In some non-limiting embodiments, tactile switch 510 may be associated with upper housing 526 and circuit board 518 and may be adjusted by a user to manually operate medical device management system 102. In some non-limiting embodiments, tactile switch 510 may be made of a material that is appropriate based on the use for which tactile switch 510 is designed. For example, the material of tactile switch 510 may include plastic, metal, rubber, elastomer, and/or the like.

In some non-limiting embodiments, emitting device 512 may include a source of electromagnetic radiation. For example, emitting device 512 may include a light-emitting diode (LED) configured to emit electromagnetic radiation in the visible light spectrum (e.g. electromagnetic radiation of wavelength 380 nm to 700 nm), an LED configured to emit electromagnetic radiation in the infrared light spectrum (e.g. electromagnetic radiation of wavelength 800 nm to 1000 nm), and/or the like. In some non-limiting embodiments, emitting device 512 may be associated with circuit board 518. In some non-limiting embodiments, emitting device 512 may be configured to emit electromagnetic radiation in the direction of a medical device associated with medical device management system 102 through an aperture of aperture plate 506. In some non-limiting embodiments, emitting device 512 may be configured to exist in an inactivated state wherein emitting device 512 does not emit electromagnetic radiation until activation by medical device management system 102. In some non-limiting embodiments, medical device management system 102 may activate emitting device 512 upon receiving data from motion sensing device 516 that exceeds a threshold. In some non-limiting embodiments, emitting device 512 may emit electromagnetic radiation upon activation by medical device management system 102.

In some non-limiting embodiments, optical reader device 514 may include an electromagnetic radiation sensor. For example, optical reader device 514 may include a photodiode (e.g., a sensor that includes a photodiode) configured to absorb electromagnetic radiation in the visible light spectrum (e.g. electromagnetic radiation of wavelength 380 nm to 700 nm), a photodiode configured to absorb electromagnetic radiation in the infrared light spectrum (e.g. electromagnetic radiation of wavelength 800 nm to 1000 nm), and/or the like. In some non-limiting embodiments, optical reader device 514 may absorb electromagnetic radiation (i.e., visible light) emitted by emitting device 512 and reflected by a medical device associated with medical device management system 102. In some non-limiting embodiments, optical reader device 514 may absorb electromagnetic radiation emitted by a medical device associated with medical device management system 102 upon absorption by the medical device of electromagnetic radiation (i.e., infrared light) emitted by emitting device 512. In some non-limiting embodiments, optical reader device 514 may be associated with circuit board 518. In some non-limiting embodiments, optical reader device 514 may provide a signal to medical device management system 102 that indicates that electromagnetic radiation of a particular wavelength has been absorbed. In some embodiments, when absorbing light in the visible light spectrum, optical reader device 514 may measure intensity of the light via a plurality of detectors (e.g., three (3) detectors) tied to fixed wavelengths. In other embodiments, when absorbing light in the infrared spectrum, optical reader device 514 may measure intensity of the light over time at the infrared wavelength. In some non-limiting embodiments, medical device management system 102 may determine that optical reader device 514 has absorbed electromagnetic radiation of a wavelength associated with a known type of medical device or an unknown type of medical device.

In some non-limiting embodiments, a medical device associated with medical device management system 102 may include a taggant configured to emit electromagnetic radiation (e.g., light) in the infrared light spectrum. The taggant comprises a uniquely encoded material or chemistry specific to the device (i.e., the taggant uniquely identifies the device). In some non-limiting embodiments, the taggant may be configured to emit electromagnetic radiation in the infrared light spectrum in an arbitrary time-decay profile corresponding to one of a plurality of known types of medical devices. In some non-limiting embodiments, data associated with optical reader device 514 may be received by medical device management system 102 and may indicate that electromagnetic radiation matching a known time-decay profile has been absorbed. In some non-limiting embodiments, medical device management system 102 may determine that optical reader device 514 has absorbed electromagnetic radiation of a time-decay profile associated with a known type of medical device or an unknown type of medical device.

In some non-limiting embodiments, motion sensing device 516 may include a sensor configured to detect motion. For example, motion sensing device 516 may include a piezoelectric sensor configured to generate data (e.g. a signal, an electrical signal, and/or the like) upon disturbance (e.g. mechanical disturbance). In some non-limiting embodiments, motion sensing device 516 may be associated with outer front section 532 and/or circuit board 518. In some non-limiting embodiments, motion sensing device 516 may generate data upon disturbance generated by association of a medical device associated with outer front section 532. In some non-limiting embodiments, medical device management system 102 may determine that data generated by motion sensing device 516 satisfies a threshold corresponding to association of a medical device.

In some non-limiting embodiments, circuit board 518 may include a printed circuit board (PCB) associated with medical device management system 102. In some non-limiting embodiments, circuit board 518 may be associated with tactile switch 510, emitting device 512, optical reader device 514, motion sensing device 516, or contact piece 522.

In some non-limiting embodiments, battery 520 may include a power storage sub-system. For example, battery 520 may include a direct current (DC) power source, one or more batteries, and/or the like. In some non-limiting embodiments, battery 520 may be associated with contact piece 522 and/or battery insulator 524. In some non-limiting embodiments, contact piece 522 may be associated with battery 520 and circuit board 518. In some non-limiting embodiments, contact piece 522 may be configured to transfer power from battery 520 to circuit board 518. In some non-limiting embodiments, contact piece 522 may be made from a material suitable for the use for which contact piece 522 is designed. For example, contact piece 522 may be made from one or more metals.

In some non-limiting embodiments, battery insulator 524 may be configured to save energy of the battery 520 during storage. In some non-limiting embodiments, battery insulator 524 may be pulled out when system 500 is to be used, so as to electrically connect battery 520 to other components/devices in the system 500 and to power up the system 500.

Referring to FIG. 5B, FIG. 5B is a partially disassembled view of a system for medical device authentication and identification 500, demonstrating that the components are designed to fit around a central axis which runs the length of deformable valve 508, allowing for fluid to pass through system for medical device authentication and identification 500, from a medical device associated with front section 504 to another medical device attached to an end of housing 502 which is not adjacent to front section 504. In some non-limiting embodiments, a needless syringe containing fluid may be associated with front section 504, and a catheter may be attached to an end of housing 502 which is not adjacent to front section 504, such that fluid from the needleless syringe may pass through deformable valve 508 to the catheter.

Referring to FIG. 5C, FIG. 5C is an assembled view of system for medical device authentication and identification 500 from the point of view of a line of sight along the central axis. As shown in FIG. 5C, housing 502 and front section 504 are configured to fit around deformable valve 508 without obstructing either end of deformable valve 508, such that fluid may pass through deformable valve 508. In some non-limiting embodiments, a medical device containing fluid may be in contact with or attached to deformable valve 508.

Referring to FIG. 5D, FIG. 5D is an assembled side view of system for medical device authentication and identification 500. As shown in FIG. 5D, housing 502, front section 504, and aperture plate 506 are configured to fit together around all other components, and specifically configured around deformable valve 508 without obstructing either end of deformable valve 508. Aperture plate 506 is configured to receive outer front section 532. Outer front section 532 is further configured to have a threaded end capable of receiving a medical device, and is further configured to receive deformable valve 508, such that an end of deformable valve 508 adjacent to outer front section 532 is unobstructed.

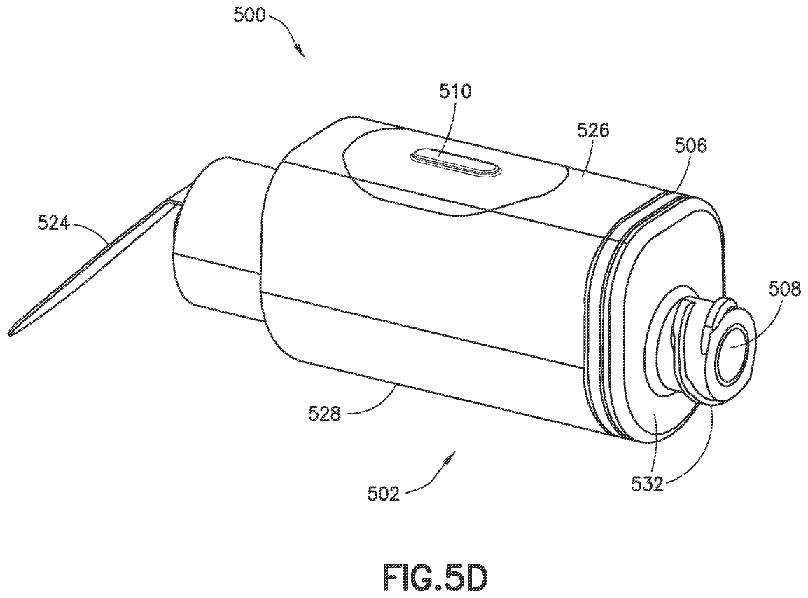

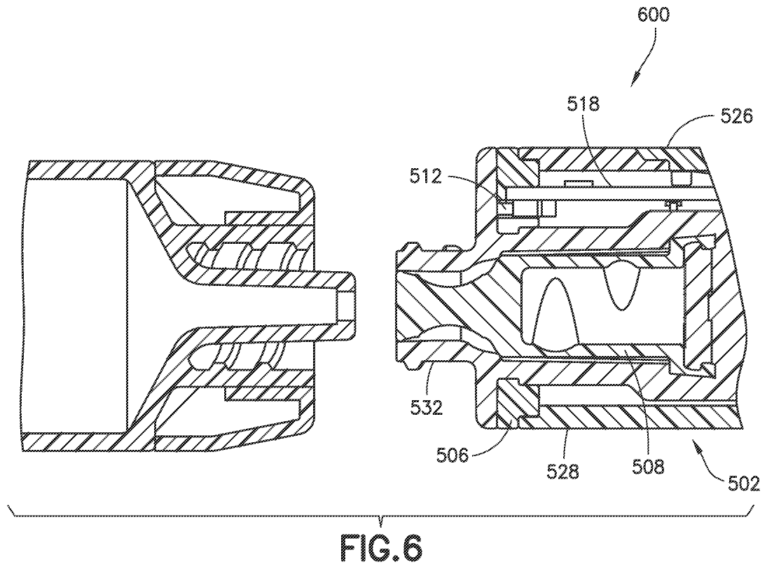

Referring to FIG. 6, FIG. 6 is a diagram of a cross-section of a non-limiting embodiment of an implementation 600 of system for medical device authentication and identification 600. In some non-limiting embodiments, system for medical device authentication and identification 600 may be associated with a medical device. For example, system for medical device authentication and identification 600 may be attached to a syringe, an IV assembly, a luer connector, a catheter, tubing, a container, medication packaging, a closure (e.g., a cap), and/or the like. As further shown in FIG. 6, system for medical device authentication and identification 600 may include an optical reader device 514 configured to absorb electromagnetic radiation reflected by or emitted by the medical device. In some non-limiting embodiments, optical reader device 514 is configured to absorb electromagnetic radiation in the visible light spectrum (i.e. having a wavelength between 380 nm to 700 nm) reflected by the medical device. In some non-limiting embodiments, optical reader device 514 is configured to absorbed electromagnetic radiation in the infrared light spectrum (i.e. having a wavelength between 800 nm to 1000 nm) emitted by the medical device.

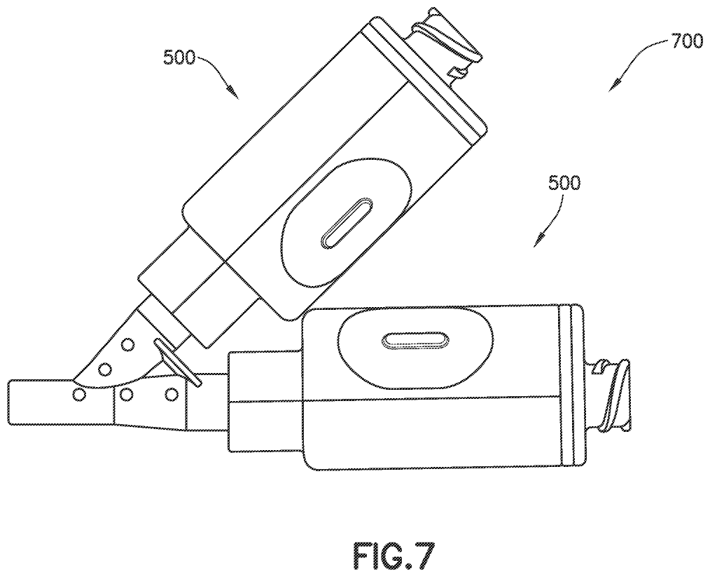

Referring to FIG. 7, FIG. 7 is a diagram of a cross-section of a non-limiting embodiment of an implementation 700 of system for medical device authentication and identification 500. As shown in FIG. 7, two systems for medical device authentication and identification 500 may be connected to a medical device. In some non-limiting embodiments, two systems for medical device authentication and identification 500 may be connected to a closed catheter connector.

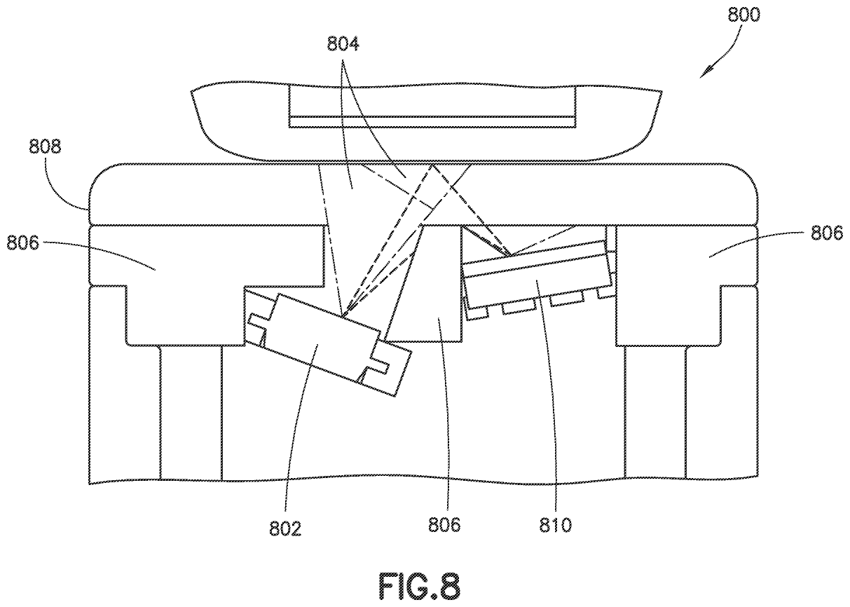

Referring to FIG. 8, FIG. 8 is a diagram of a non-limiting embodiment of an implementation 800 of system for medical device authentication and identification 500. As shown in FIG. 8, system for medical device authentication and identification 800 may include emitting device 802, electromagnetic radiation 804, aperture plate 806, outer front section 808, and optical reader device 810. In some non-limiting embodiments, emitting device 802 may be the same as or similar to emitting device 512 and/or emitting device 106. In some non-limiting embodiments, aperture plate 806 may be the same as or similar to aperture plate 506. In some non-limiting embodiments, outer front section 808 may be the same as or similar to outer front section 532. In some non-limiting embodiments, optical reader device 810 may be the same as or similar to optical reader device 514 and/or optical reader device 108.

In some non-limiting embodiments, system for medical device authentication and identification 800 may be associated with a target (e.g. a medical device, a cap, and/or the like). In some non-limiting embodiments, emitting device 802 may emit electromagnetic radiation 804 directed through an aperture in aperture plate 806 before passing through outer front section 808, which may be made clear plastic or another material that transmits radiation in the given frequencies. In some non-limiting embodiments, electromagnetic radiation 804 emitted by emitting device 802 may reflect off of the target and pass through an aperture in aperture plate 806 in the direction of optical reader device 810. In some non-limiting embodiments, aperture plate 806 limits sensitivity to ambient electromagnetic radiation with no target in place and blocks internal reflection paths. In some non-limiting embodiments, optical reader device 810 may absorb electromagnetic radiation 804 reflected off of the target. In some non-limiting embodiments, medical device management system 102 may determine a state of the medical device after optical reader device 810 has absorbed electromagnetic radiation 804 reflected off of the target.

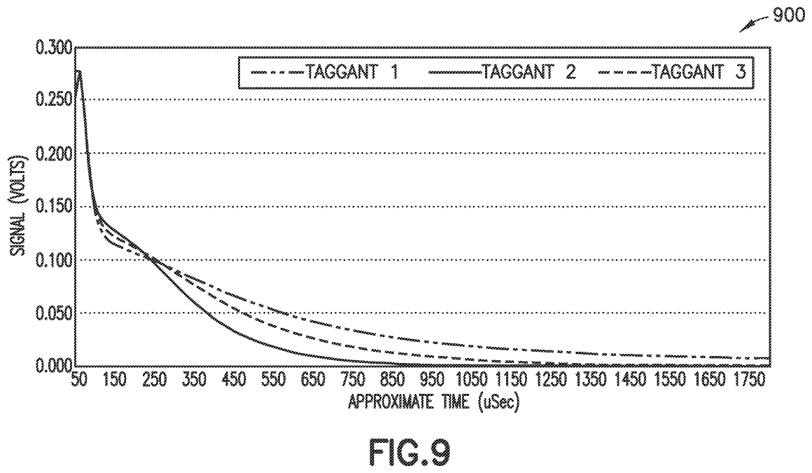

Referring to FIG. 9, FIG. 9 is a graph representing different time decay profiles of different taggants, as disclosed herein, generated by medical device management system 102. As shown in FIG. 9, three unique taggants—generically identified as Taggant 1, Taggant 2, Taggant 3—may emit electromagnetic radiation in the infrared light spectrum (i.e. electromagnetic radiation of wavelength between 800 nm and 1000 nm), with each taggant emitting electromagnetic radiation that has a distinct time-decay profile—such that three distinct time-decay profiles are provided. In some non-limiting embodiments, three taggants may be associated with three known types of medical devices. In some non-limiting embodiments, a taggant may emit electromagnetic radiation in the infrared light spectrum upon absorption of electromagnetic radiation emitted by emitting device 106. In some non-limiting embodiments, medical device management system 102 may be configured to receive data associated with a time-decay profile of a taggant.

In some non-limiting embodiments, medical device management system 102 may compare data associated with a time-decay profile of a taggant with known time-decay profiles to determine whether the state of optical reader device 108 corresponds to a known type of medical device. For example, medical device management system 102 may calculate the total area under the curve representing data regarding the electromagnetic radiation emitted by a taggant, and compare against known values corresponding to known types of medical devices. As another example, medical device management system 102 may normalize a curve representing data regarding the electromagnetic radiation emitted by a taggant at a predetermined point in time, calculate the area under the curve from the predetermined point in time, and compare against known values corresponding to known types of medical devices.

In some non-limiting embodiments, medical device management system 102 may compare data associated with a time-decay profile of a taggant with known time-decay profiles to determine whether the state of optical reader device 108 corresponds to a known type of medical device. For example, medical device management system 102 may calculate the total area under the curve representing data regarding the electromagnetic radiation emitted by a taggant, and compare against known values corresponding to known types of medical devices. As another example, medical device management system 102 may normalize a curve representing data regarding the electromagnetic radiation emitted by a taggant, calculate the area under the normalized curve to produce a value, and compare the value against stored values corresponding to known types of medical devices.

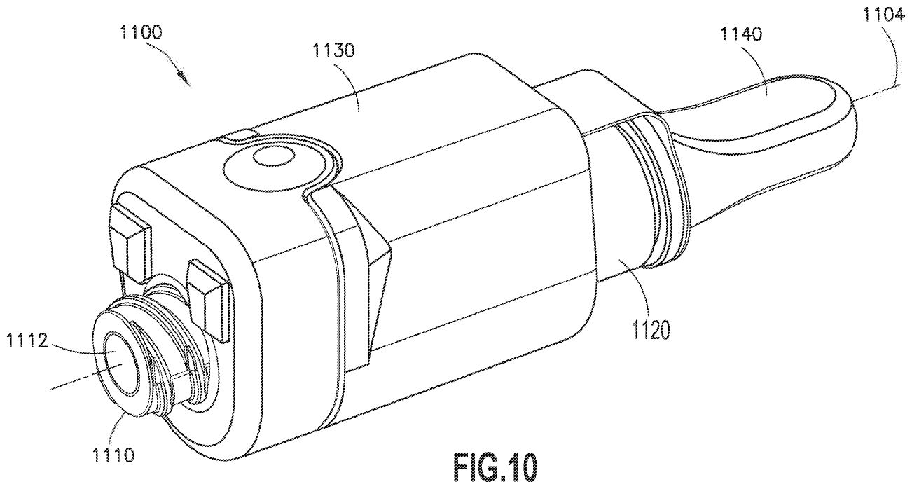

Referring now to FIGS. 10-16, an example of a needleless connector (hereinafter “connector 1100”) is illustrated in accordance with the present disclosure. Generally, the connector 1100 includes a body 1102 having a first fitting, namely a female luer fitting 1110, at one end and a second fitting, namely a male luer fitting 1120, at the opposite end. The connector 1100 may be part of a medication administration system, such as an intravenous medication administration system, that could be deployed directly on a catheter assembly, or on administration tubing or an extension set attached to that assembly. A lumen may extend through the body 1102 of the connector 1100 between the female luer fitting 1110 and the male luer fitting 1120 to facilitate fluid flow through the connector 1100. The body 1102 of the connector 1100 may be generally cylindrical in shape, defining a longitudinal axis 1104. A flexible seal 1112 is disposed in the female luer fitting 1110 and prevents contamination of the lumen by sealing the female luer fitting 1110. The female luer fitting 1110 is configured to receive a complementary male luer fitting of a component connected to the connector 1100. When connected, the complementary male luer fitting depresses the seal 1112 into the lumen, thereby opening at least one channel through the lumen to allow fluid flow through the connector 1100.

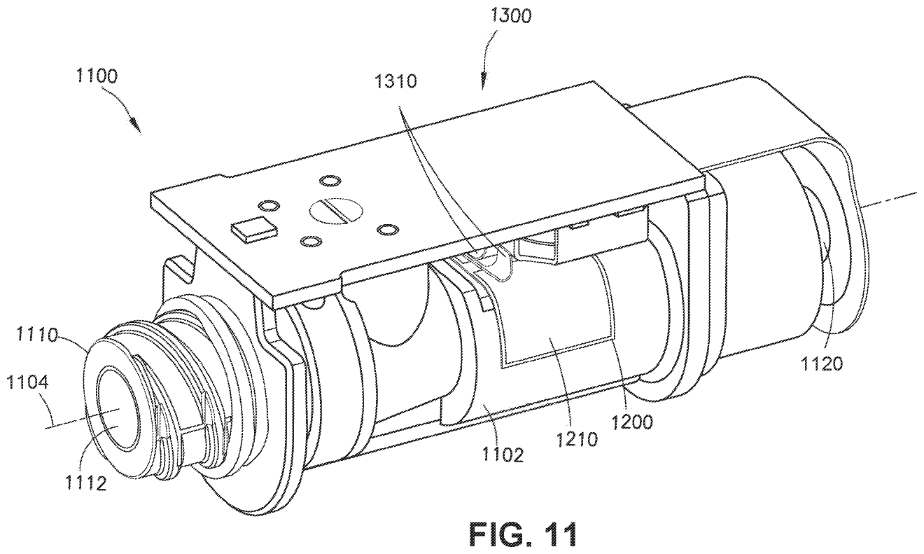

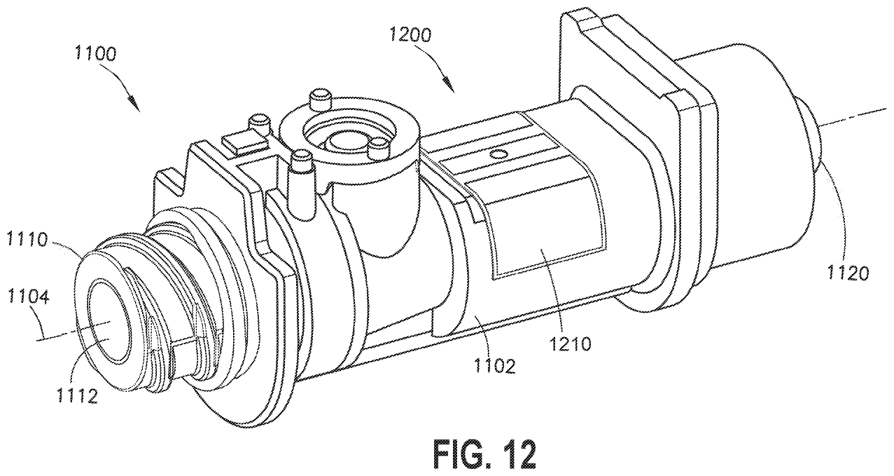

The body 1102 of the connector 1100 may be protected by a cover 1130, which may protect a scrubbing sensor 1200 and an associated electrical module 1300. The connector 1100 may further include a dust cap 1140 that covers the male luer fitting 1120 to prevent contamination of the lumen. The dust cap 1140 is removed prior to connection of the male luer fittings to another component. Removal of the dust cap 1140 allows for powering of the device by removing a protective strip from the battery, such that the device becomes powered. FIGS. 11 and 12 show the cover 1130 and dust cap 1140 removed so that underlying components of the connector 1100 can be seen.

Referring still to FIGS. 10-16, to ensure a sterile connection between the female luer fitting 1110 and a connected component (e.g. the complementary male luer fitting), the seal 1112 is disinfected prior to connection to the connected component. To disinfect the seal 1112, a clinician typically grasps the connector 1100 near the male luer fitting 1120 and scrubs the seal 1112 with a swab pad including isopropyl alcohol or another suitable disinfectant. As scrubbing is performed manually by the clinician, cleanliness and sterility of the seal 1112 is susceptible to inconsistency if left only to the skill and experience of the clinician. To eliminate such inconsistency and ensure sufficient scrubbing is performed, the connector 1100 includes the scrubbing sensor 1200 to verify that adequate force, pressure, intensity, and/or scrubbing duration have been applied during the scrubbing procedure.

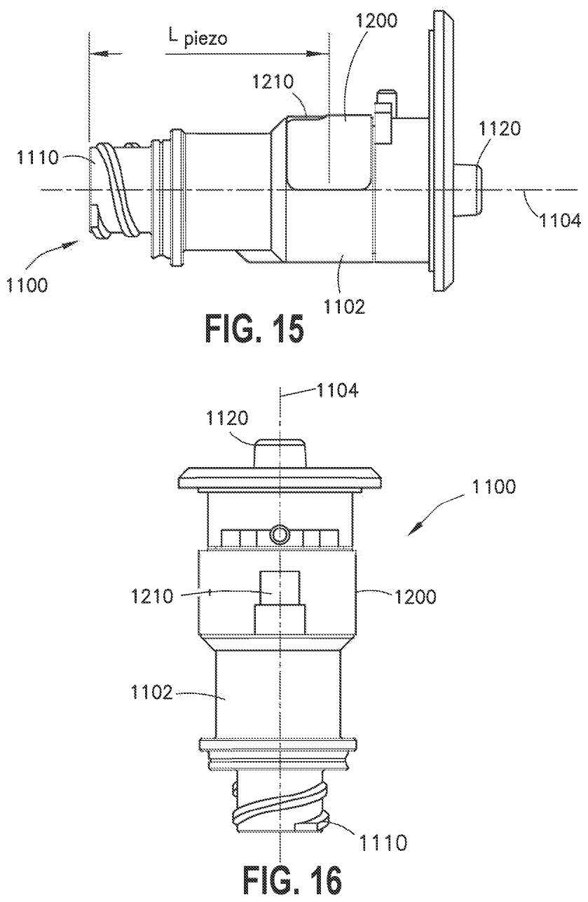

The scrubbing sensor 1200 includes a strain-to-voltage transducer, such as a piezoelectric device, configured to emit a voltage signal in response to an applied strain. The voltage signal is subsequently processed to determine, based on the voltage signal, the magnitude of a torque or force corresponding to the applied strain. In one embodiment, the scrubbing sensor 1200 includes a piezoelectric film 1210 made of, for example, polyvinylidene fluoride or polyvinylidene difluoride (PVDF). The scrubbing sensor 1200 is disposed on the body 1102 of the connector 1100, for example via an adhesive, at a predetermined distance Lpiezo from the end of the female luer fitting 1110 (as shown in FIG. 15). When a force or torque is applied to the connector 1100, the body 1102 experiences a corresponding strain. The connection between the scrubbing sensor 1200 and the body 1102 causes the stain imparted to the body 1102 to also be imparted to the scrubbing sensor 1200. As such, the voltage signal(s) emitted by the piezoelectric film 1210 in response to the strain can be processed to determine magnitude and duration of a force or torque applied to the connector 1100. By monitoring the voltage signal(s) emitted by the piezoelectric film 1210, the force and/or torque applied to the connector 1100 can be determined. Thus, the scrubbing procedure can be quantified and validated to ensure sufficient scrubbing action has been performed.

As shown in FIG. 11, the connector 1100 includes an electrical module 1300 having one or more electrical contacts 1310 that engage the piezoelectric film 1210 to receive the voltage signal from the scrubbing sensor 1200. The electrical module 1300 may then process the voltage signal itself, or transmit the voltage signal to an external processor. Based on the voltage signal and other material constants, the external process can determine scrubbing force and torque applied to the female luer fitting 1110, as described herein.

As previously stated, the clinician typically disinfects the seal 1112 by grasping the connector 1100 near the male luer fitting 1120 and scrubbing the female luer fitting 1110. When the connector 1100 is scrubbed in this manner, the static behavior of the connector 1100 can be approximated as a cantilevered cylinder. That is, the point at which the connector 1100 is held by the clinician acts as a fixed support, and the body 1102 of the connector acts as a cantilevered cylinder. The female luer fitting 1110 is unsupported, so scrubbing action applied to the female luer fitting 1110 causes a strain the body 1102 of the connector 1100 that is then imparted to the scrubbing sensor 1200. Such strain may be a result of a force applied transversely across the face of the female luer fitting 1110 (generally corresponding to a side-to-side scrubbing motion by the clinician) and/or a torque applied about an axis of the female luer fitting 1110 (generally corresponding to a circular scrubbing motion by the clinician). By detecting the strain at the location of the scrubbing sensor 1200, and known material constants, the force and torque applied to the female luer fitting 1110 can be determined with suitable accuracy to assess whether the female luer fitting 1110 and seal 1112 have been adequately scrubbed.

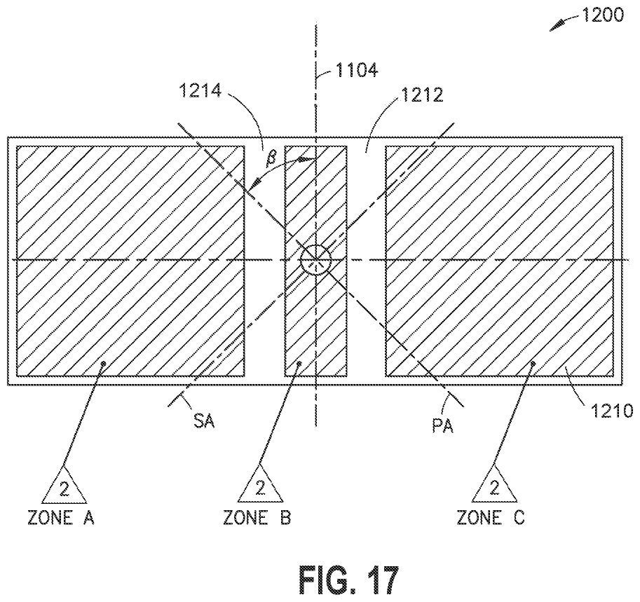

Referring now to FIG. 17-19, the scrubbing sensor 1200 may be supplied as a flat material which can be contoured to the shape of the body 1102 of the connector 1100. The piezoelectric film 1210 may have a thickness of, for example, 28 μm, and a backside of the piezoelectric film 1210 may be metalized. The piezoelectric film 1210 includes one or more contact surfaces Zone A, Zone B, and Zone C (1210 and the center strip) configured to be engaged by the electrical contacts 1310 of the electrical module 1300. As shown in FIGS. 17-19, un-metalized portions 1212 and 1214 separated the contact surfaces Zone A, Zone B, and Zone C, as shown. Each of the contact surfaces Zone A, Zone, B, and Zone C may correspond to an independent sector of the piezoelectric film 1210, which outputs an independent voltage signal in response to an applied strain. In other embodiments, the contact surfaces Zone A, Zone B, and Zone C may be electrically connected in series or in parallel so that the piezoelectric film generates a single electrical signal based on the strain at contact surfaces Zone A, Zone B, and/or Zone C. Zone A, Zone B, and Zone C are separate metalized sections as shown in FIGS. 17-19. The hole in the middle is a via which connects Zone B to the backside of the film, which is fully metalized and serves as a ground. Voltage may be formed between Ground B and separate sections A and C.

The piezoelectric film 1210 may be oriented so as to optimize detection of bending and torsional loads applied to the connector 1100. PVDF, when used as a piezoelectric film, is orientation dependent in that PVDF will generate a stronger electrical signal in response to strain along a primary axis PA, and a relatively weaker (sometimes undetectably weak) electrical signal in response to strain along a secondary axis SA orthogonal to the primary axis PA. Thus, PVDF provides the most useful and reliable response to strain that occurs in the primary axis PA. If the piezoelectric film 1210 is attached to the body 1102 of the connector 1100 such that the primary axis PA of the PVDF extends parallel to the longitudinal axis 1104 (as shown in FIG. 19), the piezoelectric film 1210 is optimized to detect bending strain in the body 1102 along the longitudinal axis 1104. Such bending strain may be caused, for example, by a bending moment induced by lateral (i.e. side-to-side) scrubbing force across the end face of the female luer fitting 1110. In this same orientation, however, the piezoelectric film 1210 may be less effective (or even functionally ineffective) at detecting torsional strain that occurs in the secondary axis SA. Such torsional strain may be caused, for example, by a circular or twisting scrubbing force on the female luer fitting 1110. Conversely, if the piezoelectric film 1210 is attached to the body 1102 of the connector 1100 such that the secondary axis SA of the PVDF extends parallel to the longitudinal axis 1104, the piezoelectric film 1210 is optimized to detect torsional strain in the body 1102 and less effective (or ineffective) at detecting bending strain in the body 1102.

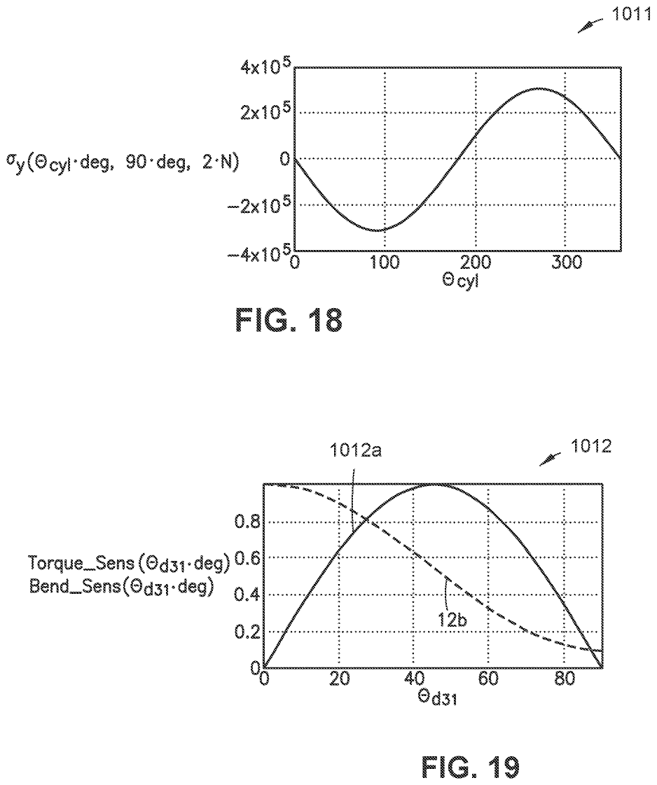

FIG. 18 illustrates a graph 1011 showing the variance in stress around the cylinder due to a single force applied in a single direction. For example, applying a load at 90 degrees puts part of the cylinder in compression, part in tension, and the parts in between under no strain.

FIG. 19 illustrates a graph 1012 showing sensitivity of the scrubbing sensor 1200 as a function of the angle β of the primary axis PA of the piezoelectric film 1210 relative to the longitudinal axis 1104. As previously indicated, applying a load at 90 degrees puts part of the cylinder in compression, part in tension, and the parts in between under no strain. Accordingly, the solid curve 1012a of graph 1012, representative of torque sensitivity, is zero when angle β is zero and/or 90°. In the illustrated embodiment, the piezoelectric film 1210 exhibits its greatest sensitivity to torsion when angle β is 45°. Conversely, and again as previously described with reference to FIGS. 17-19, the piezoelectric film 1210 may be most sensitive to bending when angle β is zero (i.e., when the primary axis PA of the piezoelectric film 1210 is parallel to the longitudinal axis 1104). Accordingly, the dashed curve 12b of graph 1012, representative of bending sensitivity, is at a maximum sensitivity when the angle ß is zero. Bending sensitivity gradually decreases as the angle β increases.

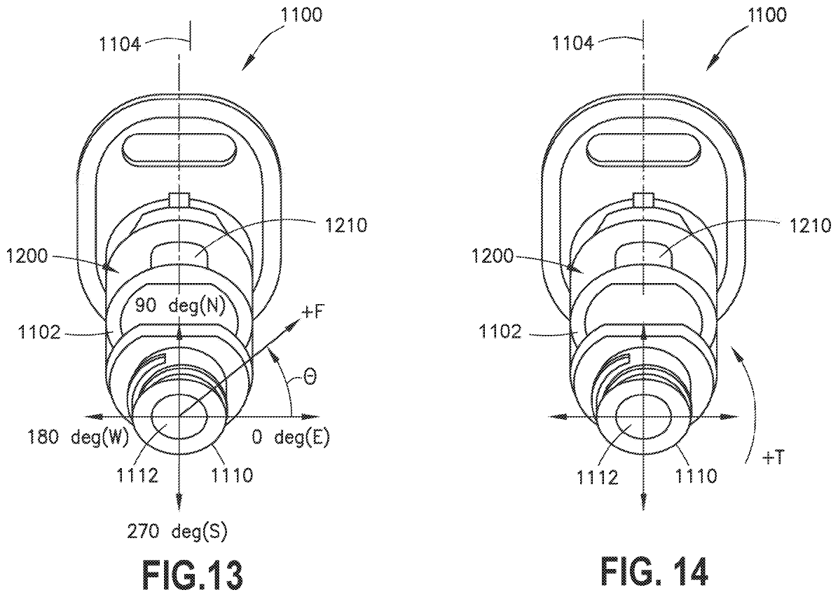

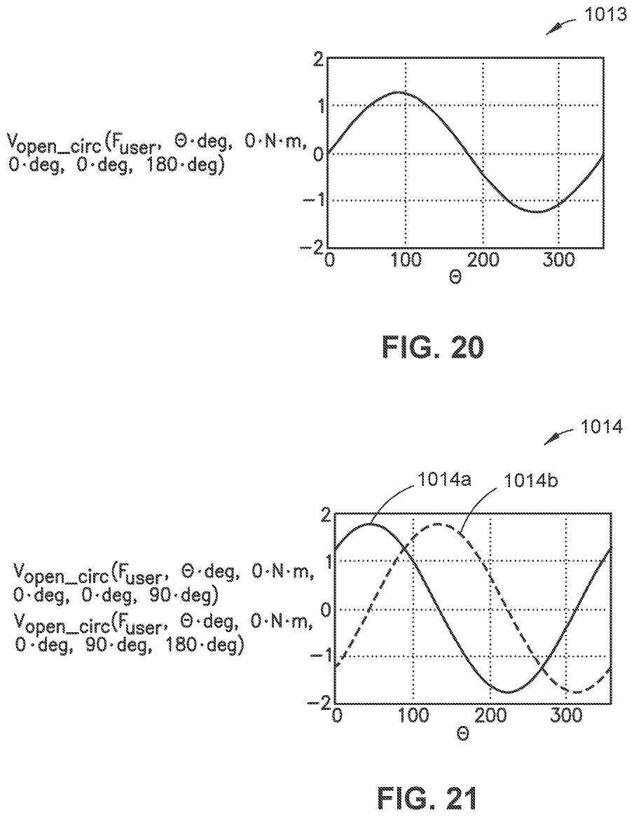

FIG. 20 illustrates a graph 1013 showing voltage output of the scrubbing sensor 1200 as a function of the angle θ at which a force F is applied to the connector 1100 (see FIG. 13 for the orientation of the angle θ and force F). In this single sector positioned symmetrically about the 90 degree position, the voltage output follows a repeating, sine-like curve, with zero voltage output when force F is applied at 0°, 180°, and/or 360°. These positions of zero voltage correspond to a null scrubbing axis, where the strain is undetectable by the scrubbing sensor 1200.

FIG. 21 illustrates a graph 1014 showing voltage output of a scrubbing sensor 1200 having two sectors arranged 90° apart about the circumference of the body 1102 of the connector 1100, as a function of the angle θ at which a force F is applied to the connector 1100. Each of the sectors is configured to output an independent voltage signal in response to strain on a corresponding area of the body 1102. First curve 1014a, representative of the voltage output of the first sector of the scrubbing sensor 1200, has the same amplitude and period as second curve 1014b, representative of the voltage output of the second sector of the scrubbing sensor 1200, but the curves 1014a and 1014b exhibit an offset period from one another. Thus, the two sectors never output zero voltage at the same angle θ, thus eliminating the null scrubbing axis. Accordingly, subdividing the scrubbing sensor 1200 into two independent sectors spaced apart from one another about the circumference of the body 1102 of the connector 1100 ensures that no direction of the force F is undetectable by the scrubbing sensor 1200. In other embodiments, the scrubbing sensor 1200 may include more than two sectors spaced apart from one another about the circumference of the body 1102.

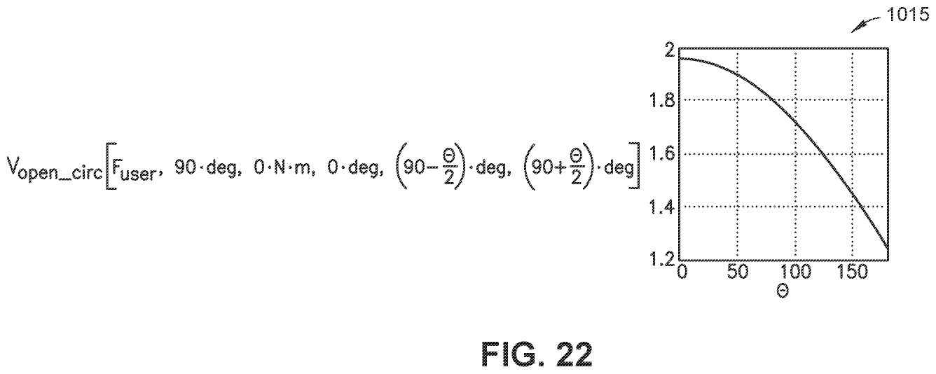

FIG. 22 illustrates a graph 1015 showing the impact of lengthening of the piezo-electric sensor. Since the sensor is applied around a cylinder with opposing strain fields, if the sensor is too long, it is counter-productive. Accordingly, the length of the sensor is to be optimized relative to the cylinder.

It is to be understood that the numerical values shown in graphs 1011-1015 are exemplary only and provided to illustrate general principles of the present disclosure. Actual embodiments of the present disclosure may exhibit different measured and/or calculated stresses, strains, voltages, etc.

Although non-limiting embodiments have been described in detail for the purpose of illustration and description, it is to be understood that such detail is solely for that purpose and that embodiments are not limited to the disclosed embodiments, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A connector for a catheter assembly, the connector comprising:
a body defining longitudinal axis and a lumen;
a first fitting at a first end of the body;
a second fitting at a second end of the body;
a flexible seal disposed in the first fitting for preventing contamination in the first fitting; and
a scrubbing sensor disposed on the body a predetermined distance from an end of the first fitting,
wherein the scrubbing sensor is configured to generate a electrical signal in response to a strain in the body, wherein the strain corresponds to at least one of a force applied to the first fitting and a torque applied to the first fitting.

2. The connector of claim 1, wherein the scrubbing sensor comprises a piezoelectric film.

3. The connector of claim 2, wherein the piezoelectric film is made of polyvinylidene fluoride.

4. The connector of claim 41, wherein the scrubbing sensor is arranged on the body such that a primary axis of the piezoelectric film is at an angle relative to the longitudinal axis.

5. The connector of claim 4, wherein the angle is approximately 30°.

6. The connector of claim 4, wherein the angle is approximately 45°.

7. The connector of claim 4, wherein the angle is in a range of approximately 10° to approximately 80°.

8. The connector of claim 4, wherein the angle is in a range of approximately 20° to approximately 70°.

9. The connector of claim 4, wherein the angle is in a range of approximately 30 ° to approximately 60°.

10. The connector of claim 4, wherein the angle is in a range of approximately 40° to approximately 50°.

11. The connector of claim 4, wherein scrubbing sensor is arranged on the body such that the primary axis of the piezoelectric film is parallel to the longitudinal axis.

12. The connector of claim 1, further comprising an electrical module comprising one or contacts configured to engage one or more contact surfaces of the scrubbing sensor to receive the voltage signal from the scrubbing sensor.

13. The connector of claim 1, wherein the scrubbing sensor comprises at least two sectors spaced apart from one another about a circumference of the body.

14. The connector of claim 13, wherein each of the at least two sectors is configured to output an independent voltage signal.

15. The connector of claim 13, wherein the at least two sectors are spaced 90° apart from one another about the circumference of the body.

16. The connector of claim 1, further comprising a cover protecting the scrubbing sensor and the electrical module.

17. The connector of claim 1, wherein the first fitting comprises a female luer fitting.

18. The connector of claim 1, wherein the second fitting comprises a male luer fitting.

19. A medical device management system comprising:
at least one processor programmed or configured to:
receive motion data associated with motion of a medical device;
determine whether the data associated with motion of the medical device satisfies a threshold;
receive optical data associated with an optical reading of a target based on determining that the data associated with motion of the medical device satisfies the threshold;
determine a state of the medical device based on the data associated with the reading of the target; and
perform an action based on the state of the medical device.

20. The medical device management system of claim 19, further comprising:
an emitting device; and
wherein the at least one processor is further programmed or configured to:
activate the emitting device based on determining that the data associated with motion of the medical device satisfies the threshold;
wherein, when receiving optical data associated with the optical reading of the target, the at least one processor is programmed or configured to:
receive optical data associated with an optical reading of a target based on activating the emitting device.

21.-38 (cancelld)
